(19) United States
(12) Patent Application Publication
(10) Pub. No.: US 2018/0235483 A1
(43) Pub. Date: Aug. 23, 2018

(54) INTEGRATED BIOSENSOR

(71) Applicant: SENSOGRAM TECHNOLOGIES, INC., Plano, TX (US)

(72) Inventor: Vahram Mouradian, Plano, TX (US)

(21) Appl. No.: 15/897,884

(22) Filed: Feb. 15, 2018

Related U.S. Application Data
(60) Provisional application No. 62/460,513, filed on Feb. 17, 2017.

(51) Int. Cl. A61B 5/0205; A61B 5/00; G06F 3/01

(52) U.S. Cl. CPC A61B 5/02055; A61B 5/681; G06F 3/017; A61B 5/021; A61B 2562/0219; A61B 2562/0271; A61B 2562/0238; A61B 2562/066

(57) ABSTRACT

A biosensor device having a housing having an inner surface, a band extending from the housing and configured to enclose an attachment site on a user's body, and a sensor package. The sensor package includes at least one optical emitter oriented to selectively emit a respective first light from the inner surface, and a plurality of optical receivers surrounding the at least one optical emitter and configured to receive reflected light at the inner surface.

This application claims priority to U.S. provisional patent application number 62/460,513 filed on Feb. 17, 2017, which is incorporated herein by reference.

### RELATED APPLICATIONS

This application is related to: U.S. application Ser. No. 15/365,242 filed on Nov. 30, 2016; U.S. application Ser. No. 14/674,499 filed on Mar. 31, 2015; U.S. provisional patent application No. 61/972,905 filed on Mar. 31, 2014; U.S. application Ser. No. 14/675,639 filed on Mar. 31, 2015; and U.S. provisional patent application No. 61/973,035 filed on Mar. 31, 2014, the disclosures of which are incorporated herein by reference for all purposes.

### TECHNICAL FIELD

The invention generally relates to wearable biosensors for detecting vital signs of the person wearing the device.

### BACKGROUND OF THE INVENTION

Biosensor systems are used to detect vital signs in the human body. These systems have been provided in a number of forms, from simple manually-operated stethoscopes and sphygmomanometers, to complex electronic monitoring systems. Early electronic biosensors were connected to the wearer and physically wired to monitoring equipment, making it difficult or impossible for the patient to move around during monitoring. More recently, electronic biosensors have been integrated into portable wearable devices that allow user mobility. For example, a typical wrist-mounted biosensor device 100 is shown in FIG. 1. This device 100 includes a housing 102 that is secured to a wearer by a band 104, and an optical sensor system 106 that faces the user's wrist. The optical sensor system 106 has an optical emitter that directs light into the wearer's wrist region, and an optical receiver that senses light reflected from the wrist region. In some cases, there may be plural optical emitters arranged around an optical emitter. A display 108, such as an interactive touchscreen or the like, is provided for observing data gathered by the optical sensor system 106. A suitable control and analysis system 110 is provided in the device 100 for controlling the optical sensor system 106 to collect vital sign data, analyzing the vital sign data, and generating the desired output. The device 100 also may include wireless communication systems, a battery, a charging port, a wired communication port, and so on.

The biosensor device 100 of FIG. 1 may be configured as a photoplethysmographic (PPG) system, which scatters light through a portion of the wearer's tissue where blood is perfused through the blood vessels (capillaries and arteries) and optically senses the absorption and/or reflection of light in such tissue. By monitoring the pulsatile change in intensity of the reflected light signal, the system can identify the wearer's heart rate and respiratory rate. A PPG system also may be configured to evaluate the absorption of multiple different wavelengths of light to evaluate blood oxygen content. It is also known, as described for example in the Applicant's copending U.S. application Ser. No. 15/365,242, to evaluate blood pressure from a PPG signal.

The biosensor device 100 may operate independently, or it may be operatively connected, such as by wired or wireless communications, to additional processors. For example, the device 100 may be wirelessly linked to a smartphone or other computer to permit remote control, processing power, and data output capabilities. The electronics and control systems for operating a biosensor device such as shown in FIG. 1 are generally known in the art, and need not be described herein in detail.

There remains a need to provide alternative techniques and systems for measuring pulse rate, blood pressure or other vital signs.

### SUMMARY

In a first aspect, there is provided a biosensor device having a housing having an inner surface, a band extending from the housing and configured to enclose an attachment site on a user's body, and a sensor package. The sensor package includes at least one optical emitter oriented to selectively emit a respective first light from the inner surface, and a plurality of optical receivers surrounding the at least one optical emitter and configured to receive reflected light at the inner surface.

The at least one optical emitter may include a plurality of optical emitters. The plurality of optical emitters may be arranged in a first square pattern, and the plurality of optical receivers may be arranged in a second square pattern surrounding the first square pattern. The plurality of optical emitters may be arranged in a first circular pattern, and the plurality of optical receivers may be arranged in a second circular pattern surrounding the first circular pattern.

A first light screen may be located between the at least one optical emitter and the plurality of optical receivers. A second light screen may surround the plurality of optical receivers.

The biosensor device may include one or more electrical sensors at the inner surface. The one or more electrical sensors may be a plurality of electrical sensors. The plurality of electrical sensors may surround the plurality of optical receivers.

The biosensor device may include one or more thermal sensors at the inner surface. The one or more thermal sensors may be a plurality of thermal sensors. The plurality of thermal sensors may surround the plurality of optical receivers.

The biosensor device may include a motion sensor and a gesture sensor.

### BRIEF DESCRIPTION OF THE DRAWINGS

Embodiments of the invention will now be described, strictly by way of example, with reference to the accompanying drawings, in which:

FIG. 1 is a representation of a prior art biosensor device

FIG. 2 is a schematic plan view of a first embodiment of a biosensor device of the invention.

FIG. 3 is a schematic plan view of a second exemplary embodiment of a biosensor device of the invention.

FIG. 4 is a schematic plan view of a third exemplary embodiment of a biosensor device of the invention.

FIG. 5 is a schematic plan view of a fourth exemplary embodiment of a biosensor device of the invention.

FIG. 6 is a schematic plan view of a fifth exemplary embodiment of a biosensor device of the invention.

FIG. 7 is a schematic elevation view of a sixth exemplary embodiment of a biosensor device of the invention.

### DESCRIPTION OF THE EMBODIMENTS

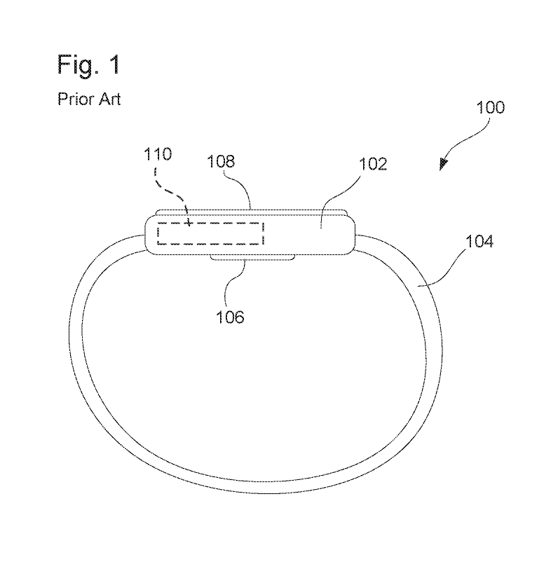

It has been found that typical wearable biosensor devices can have limitations with respect to their ability to detect blood perfusion in the underlying tissue, as well as their overall utility as a monitoring device. For example, it has been found that one problem with the prior art biosensor device 100 of FIG. 1 is that the wrist is not an ideal portion of the body for PPG analysis because it lacks the large number of detectable capillaries present in other parts of the body, such as the fingertips. In addition, wrist-mounted devices can suffer from intermittent or variable contact with the user's body.

Thus, wrist-mounted PPG devices can suffer from reduced accuracy or consistency. While these shortcoming might be overcome by moving the location of the biosensor to a more suitable observation site on the body (e.g., the fingertip), doing so can make the device 100 more cumbersome to use. Thus, it has been found that there is a need to provide improved biosensor systems for biosensor devices, and particularly for wrist-mounted biosensor devices or devices that mount at other locations on the body.

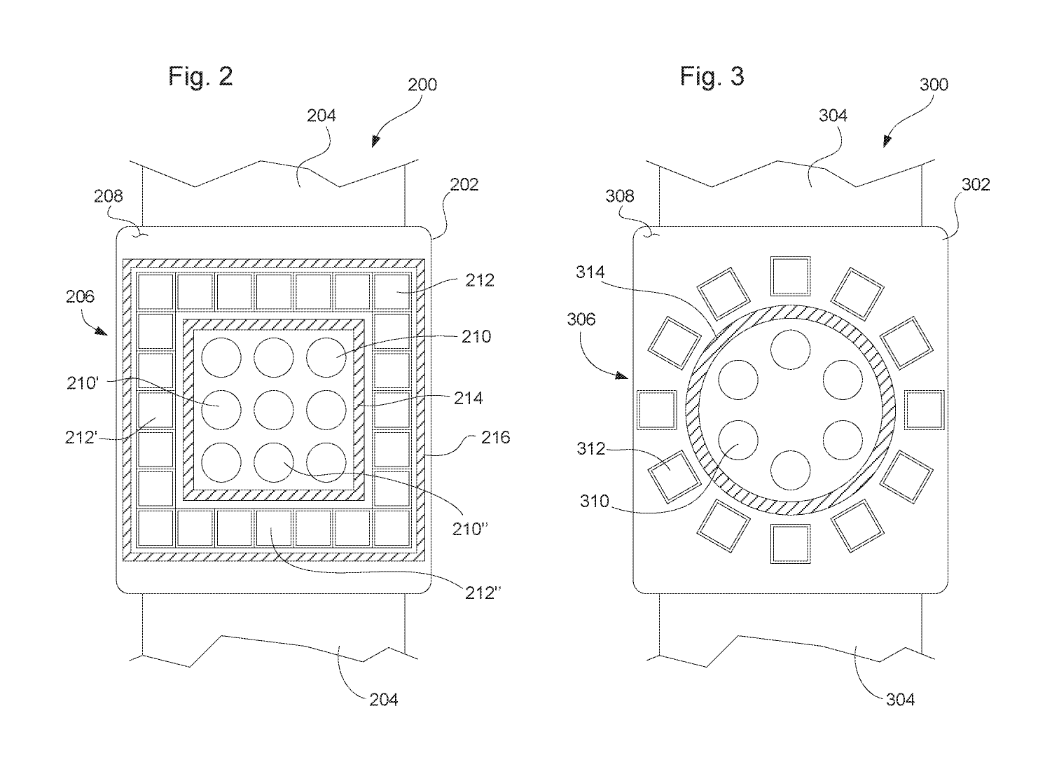

FIG. 2 illustrates a first embodiment of a biosensor device 200 having a housing 202 and a band 204. The biosensor device 200 includes a sensor array 206 facing an inner surface 208 of the housing 202 (i.e. the surface facing the user's body during use). The biosensor device 200 also may include a processing unit, a control unit, a display unit, a communication unit, and other features conventional to smart watches and wearable biosensor devices.

The housing 202 is shaped and sized to fit on the desired target location for wearing the device 200, such as the wrist. The housing 202 provides a shell or platform to which the remaining parts are directly or indirectly attached. A plastic or metallic structure is expected to be suitable for most embodiments. For example, the housing 202 may be injection-molded plastic, cast magnesium, machined aluminum or steel, or the like. The housing 202 may include surface coatings or other features such as a water-resistant shell, a glass or transparent polycarbonate face, or the like. Other alternatives will be apparent to persons of ordinary skill in the art in view of the present disclosure.

The band 204 is shown in partial view, but it will be understood that it may comprise any suitable band for securing the housing 202 to a wearer's body, such as at the wrist. For example, the band 204 may comprise two flexible or rigid straps joinable by a clasp, a single elastic strap, and so on. The band 204 may be movably or rigidly secured to the housing 202 by pivot pins, a cantilevered anchor, and so on. The band 204 also may be formed integrally with the housing 202.

When configured for use on the wrist, the housing 202 and band 204 preferably are configured with shapes and dimensions similar to a conventional wristwatch or smart watch. The housing 202 and band 204 also may comprise a conventional wristwatch or smart watch to which additional features such as discussed below are added to form an embodiment of the invention. In one example, the housing 202 may have a generally flat rectangular or rounded shape that extends in a plane with a maximum dimension in the plane of approximately two inches or less, and a thickness extending perpendicular to the plane of approximately one-half inch or less. The band 204 may be attached to edges of the housing 202 and configured to encircle a volume having a diameter of about two to three inches, or such a size as corresponds to the typical dimensions of a human wrist. The housing 202 optionally may be provided with conventional wristwatch features, such as a bezel, face and mechanical movement or digital clock for telling time.

As noted above, the sensor array 206 is located at the inner surface 208 of the housing 202. In the embodiment of FIG. 2, the sensor array 206 includes one or more optical emitters 210 located approximately centrally on the housing 202, and oriented to direct respective lights away from the inner surface 208 towards the wearer's body. The optical emitters 210 may direct the light at a 90° angle to the inner surface 208, or at an angle less than 90° thereto. In the shown example, there are nine optical emitters 210, but other numbers may be used. For example, other embodiments may have a single optical emitter 210, four optical emitters 210 in a square pattern, six optical emitters 210 in a hexagonal pattern, eight optical emitters 210 in a square or octagonal pattern, and so on. Light emitting diodes (LEDs) are preferred for use as the optical emitters 210, but other light sources may be used in other embodiments.

The optical emitters 210 may emit light at one or more wavelengths. For example, a first group of one or more of the optical emitters 210 may emit light primarily at about 350-450 nanometers (green light), a second group of one of more of the optical emitters 210 may emit light primarily at about 605-750 nanometers (red light), and a third group of the one or more optical emitters 210 may emit light primarily at about 850-1020 nanometers (infrared light). The members of each group can be clustered together, or distributed among the other groups. The different groups can be operated simultaneously or separately, as desired. For example, the red light and infrared light groups can be alternatively activated to operate in a manner to cause oxyhemoglobin and deoxyhemoglobin in the blood to absorb the different light energies, and these energy levels can be compared to determine blood oxygen saturation, using techniques known in the art.

A plurality of optical receivers 212 surround the optical emitters 210. The optical receivers 212 are oriented to receive light striking the inner surface 208, and may comprise any suitable device that is capable of determining the intensity of such light. Photodiodes, which produce a voltage or current proportional to the amount of impinging light energy, are preferred. The optical receivers 212 may form a continuous ring around the optical emitters 210, such as shown, but one or more optical receivers 212 may be emitted to provide a discontinuous ring around the optical emitters 210 (see, e.g., FIG. 3). In this example, there are twenty-four optical receivers 212 arrayed in a square pattern. Each optical receiver 212 may include a lens (e.g., a Fresnel lens) to focus light onto one or more underlying photodiodes, as known in the art. The optical receivers 212 are shown with a square shape, but this not required.

The optical receivers 212 may be tuned to detect particular wavelengths of light. For example, a first group of optical receivers 212 may have a band-pass filter that only transmits light at a range of about 350-450 nanometers (green light), a second group of optical receivers 212 may have a band-pass filter that only transmits light at a range of about 605-750 nanometers (red light), and a third group of optical receivers 212 may have a band-pass filter that only transmits light at a range of about 850-1020 nanometers (infrared light). As another example, one or more of the optical receivers 212 may include a multi-band “knife-edge” filter that allows light at multiple discrete wavelengths to pass through (e.g., a filter that transmits light at one or more wavelengths within the range of 605-750 nanometers and one or more wavelengths within the range of 850-1020 nanometers). As still another example all of the optical receivers 212 may be unfiltered.

The signal detected by any one optical receiver 212 may be conditioned in various ways. For example, where the optical receiver 212 is unfiltered or includes a knife-edge filter that transmits multiple different wavelengths, the signal from the optical receiver 212 can be demultiplexed to extract two different light signals corresponding to two different light sources being activated in an alternating sequence. Other alternatives will be apparent to persons of ordinary skill in the art in view of the present disclosure.

The arrangement of optical receivers 212 surrounding the optical emitters 210 is expected to provide benefits over conventional arrangements having a receiver surrounded by emitters. For example, this arrangement allows a greater number of optical receivers 212, which provides the opportunity to have individual or groups of optical receivers 212 specially tuned to receive individual wavelengths of light. This can increase the sensitivity of the device 200 to detecting pulsatile fluctuations in blood flow, as well as differentiating between oxyhemoglobin and deoxyhemoglobin emission profiles.

This arrangement also allows a larger number of optical emitters and receivers to be used to increase signal strength, and a significantly greater proportion of the inner surface 208 to be used for data collection, which can help avoid intermittent signal loss sometimes experienced in prior art devices. In typical wrist-mounted biosensor devices, the optical sensor package (emitter(s) and receiver(s)) take up very little of the total inner surface area of the housing. The receiver is usually close to the middle of the housing to exclude as much outside light as possible, and the emitters are located nearby to maximize the amount of reflected light that reaches the receiver. It is also believed that smaller optical sensor packages are desired amongst prior art devices to help ensure good contact with the underlying tissue; specifically, the optical sensor package is located on a small protuberance that rests firmly against the skin, and this protuberance acts like a fulcrum that remains in contact with the skin as the housing rocks through a range of motion on the wearer's body. Despite this, it has been found that many conventional devices can suffer from loss of skin contact at the optical sensor, leading to missing and erroneous data.

Placing the optical receivers 212 around the optical emitters 210 helps address this problem of inconsistent data collection by providing multiple parallel data acquisition paths that can be selectively monitored or superimposed to provide more continuous data collection. A potential data acquisition path exists between each activated optical emitter 210 and each monitored optical receiver 212. Some paths are inherently stronger than others. For example, the paths between any given optical emitter 210 and the physically closest optical receiver 212 is expected to be stronger than paths to more distant optical receivers 212 due to the reduction in signal intensity over distance according to the inverse square law. During use, however, the housing 202 is expected to move relative to the wearer's body, which can cause intermittent or sometimes continuous interruptions in the potential data acquisition paths. For example, a housing 202 may poorly conform to a wearer's wrist shape, leading to gaps between the skin and one or more optical emitters 210 or receivers 212. Such gaps can significantly reduce signal intensity and quality, and effectively eliminate the utility of one of more data acquisition paths. The use of optical receivers 212 surrounding the optical emitters 210 is expected provide at least one optical receiver 212 in contact with the body at any given time, regardless of occasional movements of the housing 202 relative to the body. Thus, it is possible to continuously monitor the desired vital signs.

The sensor array 206 can be operated and used in several different ways to take advantage of its essentially continuous sensor contact with the wearer's skin. An exemplary operation process is to: (1) activate the optical emitters 210, (2) monitor the optical receivers 212 to measure the amount of light from the optical emitters 210 that is reflected by the user's body, (3) perform signal conditioning (e.g., amplification, noise reduction and the like) on the signals received from the optical receivers 212, and (4) sum the total amplitudes of all of the optical receiver signals to arrive at a superimposed intensity value. The superimposed intensity value typically will include signal values from one or more optical receivers 212 having good contact with the skin and a good data acquisition path to one or more optical transmitters 210. Thus, the superimposed intensity value should generally provide continuous vital sign monitoring capability despite occasional shifts in the housing 202 position and mismatches with the wearer's body.

The foregoing process can be modified in a number of ways. For example, where the signal-to-noise ratio of any given optical receiver 212 is below a predetermined threshold indicating a low reliability, the output from that particular optical receiver 212 may be excluded from the superimposed intensity value. As another example, the optical emitters 210 and/or the optical receivers 212 may be activated in groups, such as groups selected according to particular light colors (e.g., alternately activating a red group and an infrared group to obtain PPG data). As another example, the values of those optical receivers 212 returning strong signals can be averaged, instead of superposed. As yet another example, the optical receivers 212 can be periodically monitored while the optical emitters 210 are not activated to determine background or baseline intensity readings.

The optical emitters 210 may be activated one at a time, or one group at a time, in series to limit the overall energy draw of the optical emitters 210. In this process, while each optical emitter 210 is activated, the nearby optical receivers 212 may be monitored, while the more remote optical receivers 212 that are not likely to receive a useable signal are not monitored.

The device 200 also may selectively discontinue use of certain optical emitters 210 or optical receivers 212 that are consistently failing to provide useable results. For example, the device 200 can perform a data acquisition path quality test by activating the optical transmitters 210 and monitoring the optical receivers 212 to determine which optical receivers 212 are receiving the strongest reflected light signals. This test can be modified by cycling the operation of individual optical emitters 210 to more specifically identify which of the optical emitters 210 are responsible for providing the majority of the reflected light signals received at the optical receivers 212. For example, using this process, the device 200 might determine that there is a strong data acquisition path between a first optical emitter 210′ and a first optical receiver 212′, but a weak data acquisition path between a second optical emitter 210″ and a second optical receiver 212″. After making this determination, the device 200 can be operated by ceasing activation of the second optical emitter 210″ and/or monitoring of the second optical receiver 212″ for a certain number of cycles to avoid expending energy on an inefficient or ineffective data acquisition path. After the predetermined number of cycles is complete, the device 200 can once again perform the data acquisition path quality test described above to determine whether a good data acquisition path has been established between second optical emitter 210″ and second optical receiver 212″ due to movement of the housing 202 on the wearer's body or the like.

The use of a diagnostic quality test such as the foregoing is expected to reduce energy consumption and may improve operation accuracy. This process also effectively actively customizes the sensor array 206 to work with the particular contours of the wearer's body and the circumstances of use. This is expected to provide a significant improvement over conventional biosensor devices that rely on detecting vital signs at what is essentially a single observation point on the body.

The sensor array 206 may include one or more light screens 214, 216 on the inner surface 208 of the housing 202. In this example, a first light screen 214 is located between the optical emitters 210 and the optical receivers 212. The first light screen 214 comprises a material that is opaque to the wavelengths of light emitted by the optical emitters 210, and operates to prevent light from passing directly from the optical emitters 210 to the optical receivers 212. The first light screen 214 may extend a short distance from the housing's inner surface 202, and may comprise a relatively soft material, such as rubber or flexible polymer, to rest gently on the wearer's skin and generally maintain contact with the skin as the housing 202 moves relative to the body. The first light screen 214 may comprise a continuous part or multiple segments that overlap or join to form a continuous light barrier around the optical transmitters 210, but this is not strictly required. The second light screen 216 surrounds the optical receivers 212 to help prevent stray light from impinging on the optical receivers 212. The second light screen 216 may be similar to the first light screen 214, or have a different construction. The second light screen 216 also may be omitted (see, e.g., FIG. 3).

The arrangement shown in FIG. 2 has a square pattern of optical emitters 210 and a corresponding square pattern of optical receivers 212. This configuration is expected to greatly increase the overall surface area usage of a conventional smart watch or the like. For example, the outermost periphery of the sensor array 206, as defined by the area within the outermost periphery of the optical receivers 212, may cover about 65% or more of the total inner surface 208 of the housing 202.

Other embodiments may have other configurations of optical receivers surrounding optical emitters. For example, FIG. 3 illustrates another exemplary embodiment of a biosensor device 300 having a housing 302, a band 304, and a sensor array 306 located at the inner surface 308 of the housing 302. The sensor array 306 has a plurality of optical emitters 310 arranged in a circular array, and a plurality of optical receivers 312 arranged in a circular array surrounding and centered on the circular array of optical emitters 310. Light screens also may be provided, such as a circular light screen 314 between the optical emitters 310 and optical receivers 312. In this embodiment, the optical receivers 312 are positioned at a more uniform distance from the nearest optical emitter 310, than in the example of FIG. 2 (note that the optical receivers 212 in the corners of the square array are relatively far from any optical emitter 210), which may provide more uniform results among the optical receivers 312. This is not, however, strictly required.

It will be readily appreciated that other geometric patterns of optical emitters and optical detectors may be used in other embodiments.

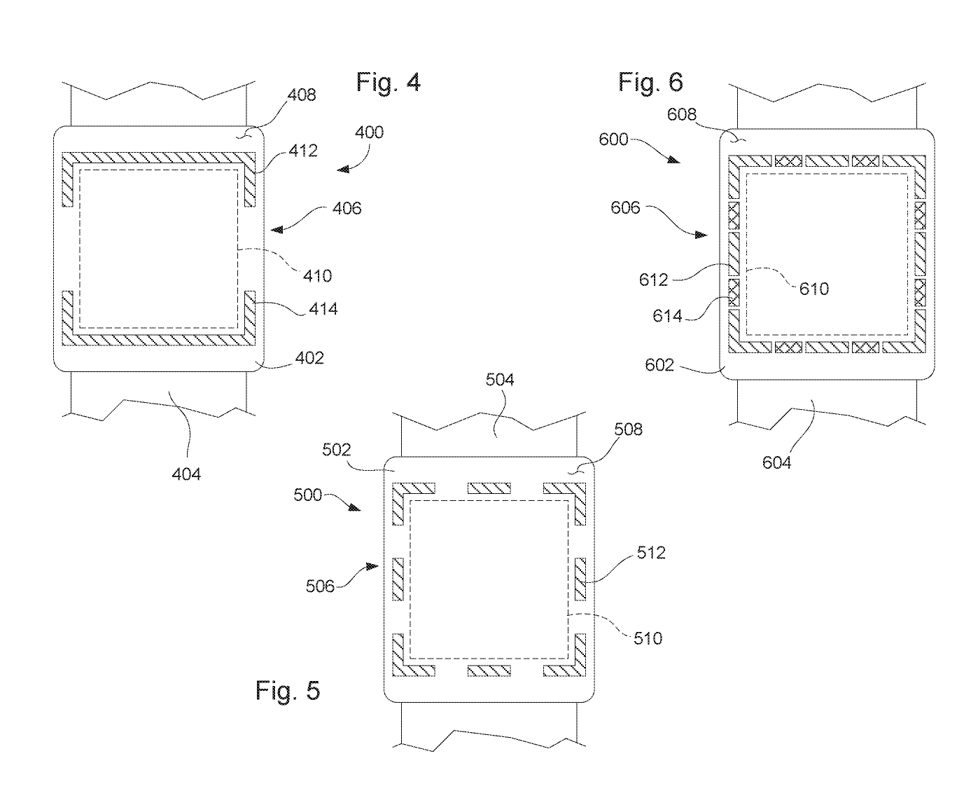

FIG. 4 illustrates a further embodiment of a biosensor device 400 having a housing 402, a band 404, and a sensor array 406 located at the inner surface 408 of the housing 402. The sensor array 406 includes an optical sensor 410 such as those described in the embodiments of FIGS. 2 and 3 (i.e., one or more optical emitters and one or more optical receivers preferably surrounding the optical emitter(s)). The optical sensor 410 portion of the sensor array 406 is shown in outline in FIG. 4. The sensor array 406 also includes one or more electrical sensors, such as first and second dry electrode contacts 412, 414, located on the inner surface 408 of the housing 402. The electrode contacts 412, 414 are configured to contact the wearer's skin, and are electrically connected to a processor that is configured with a monitoring circuit to analyze the body's electrical behavior.

A number of different electrical monitoring circuits may be employed with the electrode contacts 412, 414. For example, the processor may comprise a galvanic skin response circuit that is configured to transmit an electric stimulus through the electrode contacts 412, 414 to the skin, and measure the skin's reaction to assess variations in electrical conductance that might be representative of certain physiological conditions. In this configuration, the first and second contacts 412, 414 may comprise two ends of an open circuit that is completed when both contacts 412, 414 touch the wearer's skin.

The electrode contacts 412, 414 also may be connected to a circuit that measures electrical impulses generated by the user's muscles (including the heart) to evaluate heart rate and other body activity. In this case, the electrode contacts 412, 414 may be two open ends of a monitoring circuit. Alternatively, the electrode contacts 412, 414 may be wired in parallel as two parallel branches of a common electric circuit node, and a separate electrode contact (not shown) may be provided remotely to close the electric circuit. For example, a remote electrode contact forming the other end of the circuit may be connected by a wire or wirelessly to the biosensor device 400 (e.g., an electrode provided in a band worn on the other wrist or on the chest). In this way, the biosensor device 400 can monitor electrical impulses passing through larger portions of the wearer's body (e.g., wrist-to-opposite wrist or chest-to-wrist). The use of multiple parallel electrode contacts 412, 414 allows redundant connections in case one contact loses contact with the wearer's skin. Alternatively, a single electrode contact may be provided on the housing 402 if such redundancy is not desired.

Operative features of electrical monitoring devices having contacts through which electrical properties of the body are detected (e.g., circuit design and the like) are generally known, and need not be described in greater detail herein.

FIG. 5 illustrates a further embodiment of a biosensor device 500 having a housing 502, a band 504, and a sensor array 506 located at the inner surface 508 of the housing 502. As with FIG. 4, the optical sensor 510 portion of the sensor array 506 is shown in outline, but it may comprise any configuration of optical emitters and optical receivers preferably surrounding the optical emitters. In this embodiment, the sensor array 506 includes a plurality of electrode contacts 512 (shown as the shaded portions) arranged around the outer perimeter of the optical sensor 510. The electrode contacts 512 provide multiple redundant data acquisition paths for collecting electrical information from the wearer's body. The electrode contacts 512 may all be wired in parallel to form on open end of an electrical circuit (e.g., a heart impulse monitoring circuit), and a separate remote contact may be placed elsewhere on the wearer's body to provide the other open end of the electrical circuit. Alternatively, the electrode contacts 512 may be wired in multiple parallel groups, with each group being one end of an electric circuit. For example, four of the illustrated eight electrode contacts 512 may be connected in parallel with one another and comprise one open end of an electric circuit (e.g., a galvanic skin response circuit), and the remaining four electrode contacts 512 may be connected in parallel with one another and comprise a second open end of the electric circuit. In either case, the wearer's body closes the open ends of the electric circuit to provide vital sign monitoring.

Again, the use of multiple electrode contacts 512 offers redundancy among the potential data acquisition paths. The biosensor device 500 may be programmed to perform periodic diagnostic quality tests to determine which of the electrode contacts 512 are providing useable data. For example, the electrode contacts 512 may be cycled in pairs to determine which pairs are joined electrically by the wearer's body, and electrode contacts 512 that do not indicate a suitable electrical circuit with the wearer's body can be excluded from further use for a period of time to conserve battery resources.

It will be appreciated that the electrode contacts of the foregoing embodiments may be modified in a number of ways. For example, any number of electrode contacts may be used, and they may be located at any suitable location. Also, a position outside the optical sensor portion of the sensor array is preferred, but not required. For example, one or more electrode contacts may be located at the center of or elsewhere within the optical sensor portion. As one example, one or more of the optical emitters or optical receivers of FIGS. 2 and 3 may be replaced by an electrode contact.

One or more electrode contacts also may be configured as a light screen, such as those shown in the embodiments of FIGS. 2 and 3. For example, the first light screen 214 may be one electrode contact and the second light screen 216 may be a second electrode contact. As another example, the second light screen 216 may comprise a first portion formed by a first electrode contact and a second portion formed by a second electrode contact, and the two electrode contacts may be spaced by electrical insulating material forming the remainder of the light screen. Other alternatives will be apparent to persons of ordinary skill in the art in view of the present disclosure.

FIG. 6 illustrates a further embodiment of a biosensor device 600 having a housing 602, a band 604, and a sensor array 606 located at the inner surface 608 of the housing 602. This embodiment also includes an optical sensor 610, which is again shown in outline. The biosensor device 600 optionally also includes one or more electrode contacts 612 (shown as shaded regions), which may be connected to a suitable electrical circuit for monitoring the wearer's body. The sensor array 606 further includes one or more thermal sensors 614 (shown as crosshatched regions). The thermal sensors 614 are configured to detect heat at the wearer's skin. Any suitable temperature-measuring device may be used, such as a “Seebeck” type heat flux sensor (such as those available from greenTEG™ of Zurich, Switzerland), a thermocouple, a thermistor, a resistance temperature detector (“RTD”), an integrated silicon-based sensor, and so on. Such devices generate a voltage or current value that is proportional or otherwise related to the temperature of the sensor or the amount of heat passing through the sensor.

While a single thermal sensor 614 may be used, it is preferred to have multiple thermal sensors 614 located at various locations on the inner surface 608 to provide a more robust sampling of the wearer's body. To this end, the processor operating the biosensor device 600 may evaluate the outputs from the multiple thermal sensors 614 to determine which of the thermal sensors 614 may or may not be in contact with the wearer's skin, such as by excluding temperature readings that are outside the normal skin temperature range for the part of the body where the housing 602 is located, and averaging the values of all of the temperature readings that are within the normal skin temperature range at that location. The thermal sensors 614 also may be observed for dramatic changes in temperature over time, which can indicate that a thermal sensor 614 is making intermittent contact with the wearer's skin, and those exhibiting such behavior excluded from analysis until they provide a more consistent temperature reading. Other alternatives will be apparent to persons of ordinary skill in the art in view of the present disclosure.

The thermal sensors 614 may be used to monitor local body temperature, from which a core temperature can be surmised via lookup tables, calibration data, or the like. If the thermal sensors 614 are positioned where they can be affected by temperature changes caused by operation of the optical emitters (e.g., heated by the optical emitters when the optical emitters are active), a correction factor may be applied during such operation. However, it is more preferable for the thermal sensors 614 to be thermally insulated from any parts that might cause a significant temperature increase not associated with the changes in the wearer's body temperature.

The thermal sensor(s) 614 may be distributed at any location where they can contact the wearer's skin. As shown in FIG. 6, the thermal sensors 614 may be interposed between a plurality of electrode contacts 612. The thermal sensors 614 also may be located among the optical sensor 610 components, and may be configured to form a functional structure such as a light screen. Other alternatives will be apparent to persons of ordinary skill in the art in view of the present disclosure.

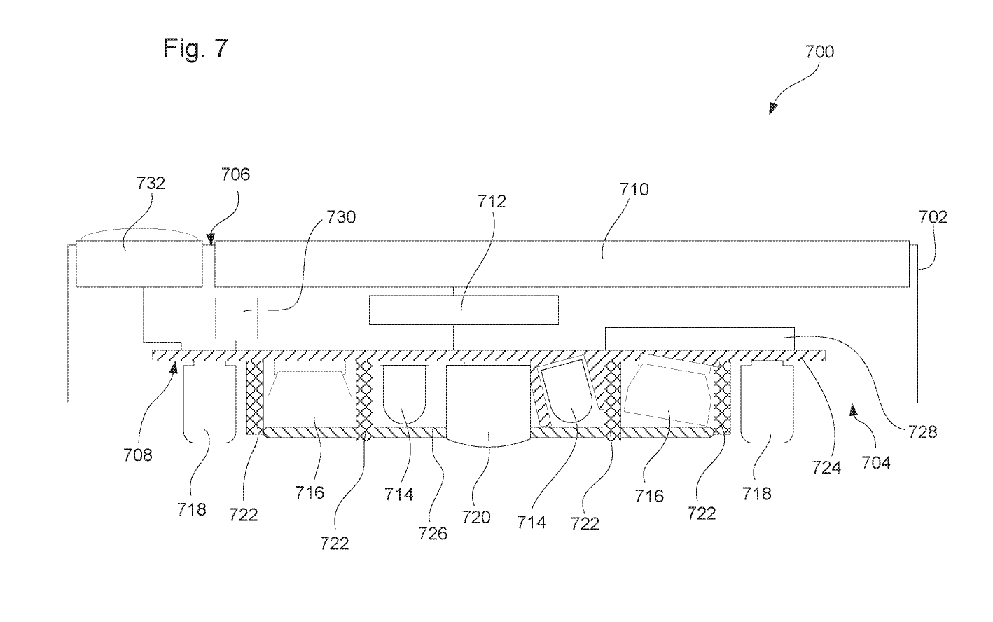

FIG. 7 illustrates another embodiment of a biosensor device 700. This biosensor device 700 has a housing 702 with an inner surface 704 that faces the wearer and an outer surface 706 that faces away from the wearer. An integrated sensor package 708 is located at the inner surface 704, and an interface 710 is located at the outer surface 706. A band (not shown) is attached to the housing for securing the housing 702 to the wearer's body. A processor 712 is electrically connected to the sensor package 708 for control thereof. The processor 712 may include any suitable microprocessor or collection of microprocessors or electronic components. An ultra-low power microprocessor is preferred. In one embodiment, the processor 712 may be based on the 32bit ARM Cortex-M4 core, which includes a variety of peripheral devices. The microprocessor may have an ultra-low power consumption of about 238 μA/MHz in dynamic run mode, and 0.35 μA in lowest power mode. The processor 712 preferably has sufficient power and speed to allow essentially continuous collection and processing of data from the various sensors, but it may be configured to alternate between data collection and data processing stages to conserve power consumption. The details of processors and peripheral devices that can be used in a wearable biosensor devices are generally known in the art, and need not be described in detail herein.

The integrated sensor package 708 includes one or more optical emitters 714, one or more optical receivers 716, one or more electrode contacts 718, and one or more thermal sensors 720. The optical emitters 714 preferably are surrounded by the optical receivers 716, and one or more light screens 722 may be provided to block direct transmission of light from the optical emitters 714 or the outside environment to the optical receivers 716. The optical emitters 714 and optical receivers 716 may be oriented normal to the inner surface 704 (as shown on the left side of the illustration), or they may be angled at less than 90° to the inner surface 704 (as shown on the right side of the illustration). Angling the optical emitters 714 and optical receivers 716 towards a common point can provide a benefit by concentrating the emitted light energy at a location more directly observed by the optical receiver 716. The optical emitters 715 and optical receivers 716 also may include collimators or lenses to more narrowly concentrate the emitted light energy and the observed region of the body.

The optical emitters 714, optical receivers 716, electrode contacts 718 and thermal sensors 720 preferably are integrated into a single sensor package 708 such as by mounting all of these components on a common platform such as a circuit board, chip or subhousing. In this case, the aforementioned components are connected by a common board 724, to which the light screens 722 are also may be connected. A transparent cover 726 also may be attached to the assembly to contain and protect the optical emitters 714 and optical receivers 716. The thermal sensor 720 and electrode contacts 718 may protrude through or be located outside the periphery of the cover 726 to allow more direct contact with the wearer's skin. The thermal sensor 720 and electrode contacts 718 also may protrude somewhat from the surface of the cover 726 to help provide more intimate contact with the wearer's skin at these points. The integrated sensor package 708 also may include a dedicated integrated sensor package processor 728 for controlling the sensors and managing output signals prior to delivery of the signals to the processor 712.

The biosensor device 700 also may include additional sensors, such as a motion sensor 730 and a gesture sensor 732. These additional sensors can be integrated directly onto the sensor platform with the other sensors, or provided as separate sensors that are electrically connected to the main processor 712 or sensor package processor 728.

The motion sensor 730 is used to track the wearer's movement, which may be helpful to determine the wearer's physical state at the time certain vital signs are measured. For example, changes in respiration, blood oxygen content, pulse rate and blood pressure can be correlated to certain physical activity or lack thereof. In addition, sudden changes in such vital signs during a physically-idle period might indicate a medical condition, such as cardiac arrest. The motion sensor 730 may comprise an inertial motion sensing instrument, such as a 6-axis motion sensor having a 3-axis gyroscope and a 3-axis accelerometer, or a 9-axis motion sensor having a 3-axis gyroscope, a 3-axis accelerometer, and a 3-axis magnetometer. Such devices typically have integrated microcontrollers and the like to facilitate integration into a wide variety of applications. Exemplary products are available from Bosch-Sensortec GmbH or Reutlingen, Germany.

The exact location of the motion sensor 730 within the housing 702 may be modified as desired. It is not expected that any particular location will provide unique benefits over other locations with relation to the operation of the motion sensor 730 or other sensors, because motion sensors 730 generally do not require access to observe outside environments and are not expected to be particularly susceptible to electromagnetic interference caused by other parts. However, in the shown embodiment, the optical, thermal and electrical sensors are interposed between the motion sensor 730 and the inner surface 704 of the housing 702 in order to maximize the size of the optical, thermal and electrical sensor portion of the package relative to the inner surface 704. Maximizing the size of the optical portion of the sensor package 708, in particular, is expected to provide certain benefits as noted above.

The gesture sensor 732 is used to observe the surrounding environment to receive gesture-based control inputs from the user, and thus the gesture sensor 732 is mounted at the outer surface 706 of the housing 702. The shown embodiment is on the portion of the outer surface 706 facing opposite the inner surface 704. In other embodiments the gesture sensor 732 may be located on a sidewall portion of the outer surface 706 of the housing 702, or at other suitable locations. The gesture sensor 732 may be mounted directly to the integrated sensor package 708 platform, or mounted separately to the housing 702 and electrically connected to the main processor 712 or the sensor package processor 728. Other alternatives will be apparent to persons of ordinary skill in the art in view of the present disclosure.

The gesture sensor 732 is programmed to identify and interpret certain physical movements, such as finger and hand movements of a nearby hand. Such input can be used to perform operations such as powering the device 700 on and off, beginning or ending data acquisition, accessing menus, reviewing data, and so on. Such devices are known in the art, and can include red/green/blue color sensor, infrared sensors, active lighting to assist with operation in low light conditions, and a dedicated processor for facilitating integration into other circuits. It is expected that suitable sensors can be based on the APDS-9960 chip from Broadcom Limited (formerly Avago Technologies) of San Jose, Calif.

The interface 710 can include any suitable collection of input and output devices, such as one or more buttons and electronic (e.g., LCD or LED) displays, or an interactive touchscreen that functions to receive input and provide output. The gesture sensor 732 also can act as an aspect of the interface 710, and it may be electrically connected directly to a dedicated interface processor if one is provided. The interface 710 is electrically connected to the processor 712 for operation therewith, as known in the art.

The biosensor device 700 also may include various other features, such as data and power input and output ports (e.g., micro USB), wireless communication modules (e.g., Bluetooth, Near Field Communication, Zigbee, etc.), one or more batteries, and so on. The biosensor device 700 also may include the functional features of a wristwatch or a smart watch.

It is envisioned that the various embodiments can be modified in a number of ways. For example, features of one embodiment can be combined with or substituted for features of other embodiments. It is also envisioned that one or more of the sensors (optical, electric, thermal, motion or gesture) can be moved to the band or to another location remote from the housing. For example, the inner surface of the device might be the inner surface of the band, rather than the inner surface of the housing, or a second housing may be provided elsewhere on the band. The housing and band also may be reconfigured for use at locations other than the wrist (e.g., the upper arm, waist, chest or leg), and for use on non-human subjects. Other alternatives will be apparent to persons of ordinary skill in the art in view of the present disclosure.

Embodiments may be used in a number of ways. For example, a biosensor as disclosed herein can be used to monitor heart rate, respiration rate, oxygen saturation, blood pressure, body temperature, skin galvanic conditions, electrical impulses reflective of pulse rate or muscular contraction, body movement, and so on. The sensors may be operated continuously or intermittently (e.g., on demand or at predetermined intervals). The device also may be remotely operated to perform remote data collection and analysis. For example, a patient undergoing medical care can be monitored remotely by a doctor that initiates remote data collection and review wirelessly though the internet or cellular networks.

The present disclosure describes a number of new, useful and nonobvious features and/or combinations of features that may be used alone or together. The embodiments described herein are all exemplary, and are not intended to limit the scope of the inventions. It will be appreciated that the features shown and described in documents incorporated herein by reference may be added to embodiments in a manner corresponding to the use of such features in the incorporated references. It will also be appreciated that the inventions described herein can be modified and adapted in various ways, and all such modifications and adaptations are intended to be included in the scope of this disclosure and the appended claims.

1. A biosensor device comprising:
a housing having an inner surface;
a band extending from the housing and configured to enclose an attachment site on a user's body; and
a sensor package comprising:
at least one optical emitter oriented to selectively emit a respective first light from the inner surface, and
a plurality of optical receivers surrounding the at least one optical emitter and configured to receive reflected light at the inner surface.

2. The biosensor device of claim 1, wherein the at least one optical emitter comprises a plurality of optical emitters.

3. The biosensor device of claim 2, wherein the plurality of optical emitters is arranged in a first square pattern, and the plurality of optical receivers is arranged in a second square pattern surrounding the first square pattern.

4. The biosensor device of claim 2, wherein the plurality of optical emitters is arranged in a first circular pattern, and the plurality of optical receivers is arranged in a second circular pattern surrounding the first circular pattern.

5. The biosensor device of claim 1, further comprising a first light screen located between the at least one optical emitter and the plurality of optical receivers.

6. The biosensor device of claim 5, further comprising a second light screen surrounding the plurality of optical receivers.

7. The biosensor device of claim 1, further comprising one or more electrical sensors at the inner surface.

8. The biosensor device of claim 7, wherein the one or more electrical sensors comprises a plurality of electrical sensors.

9. The biosensor device of claim 8, wherein the plurality of electrical sensors surrounds the plurality of optical receivers.

10. The biosensor device of claim 1, further comprising one or more thermal sensors at the inner surface.

11. The biosensor device of claim 10, wherein the one or more thermal sensors comprises a plurality of thermal sensors.

12. The biosensor device of claim 11, wherein the plurality of thermal sensors surrounds the plurality of optical receivers.

13. The biosensor device of claim 1, further comprising a motion sensor.

14. The biosensor device of claim 1, further comprising a gesture sensor.

15. The biosensor device of claim 1, further comprising:
one or more electrical sensors at the inner surface;
one or more thermal sensors at the inner surface;
a motion sensor; and
a gesture sensor.
